(19) United States
(12) Patent Application Publication
(10) Pub. No.: US 2025/0009267 A1
(43) Pub. Date: Jan. 9, 2025

(54) CVC LINE BLOOD DRAWING ASSEMBLY FOR NON-STERILE USE

(71) Applicant: JASON HOLLIS, CLIO, MI (US)

(72) Inventor: JASON HOLLIS, CLIO, MI (US)

(21) Appl. No.: 18/890,655

(22) Filed: Sep. 19, 2024

Related U.S. Application Data
(63) Continuation-in-part of application No. 17/151,107, filed on Jan. 15, 2021.
Application No. 17/151,107 is a division of application No. 16/053,544, filed on Aug. 2, 2018, now abandoned.

(51) Int. Cl. A61B 5/15; A61M 25/00

(52) U.S. Cl. CPC A61B 5/150992; A61B 5/150267; A61B 5/150328; A61M 25/0097

(57) ABSTRACT

A blood drawing assembly for use with a CVC hub includes a collection hub with a plurality of ports for the acceptance of a plurality of syringes. A CVC hub is coupled to the collection hub and routed internally within the collection hub to a syringe attachment device. The syringe attachment device is selectively moved to align with one of a plurality of syringes for the flushing of the CVC and the withdrawing of the blood. The assembly is used in non-sterile environments and is configured to be disposable after use. Fluids may be exchanged or passed through the collection hub and the syringes from the patient's body.

### CROSS REFERENCE TO RELATED APPLICATIONS

This application claims the benefit of an earlier filing date and right of priority to U.S. Non Provisional patent application Ser. No. 17/151,107, filed 15 Jan. 2021 which is a divisional from U.S. Non Provisional application Ser. No. 16/053,544, filed 2 Aug. 2018, the contents of which is incorporated by reference herein in its entirety.

### BACKGROUND OF THE INVENTION

### 1. Field of the Invention

The present application relates to an assembly for the capture of blood from a patient, and more particularly to a disposable and sterile multi syringe assembly for the capture of blood while reducing the ability of bacteria and virus introduction to enter a central venous catheter along with reducing blood waste.

### 2. Description of Related Art

A central venous catheter (CVC) is also known as a central line, central venous line, or central venous access catheter. A CVC is a catheter that is placed into a large vein of the body, typically around the neck (internal jugular vein), chest (subclavian vein or axillary vein), groin (femoral vein), or through veins in the arms (also known as a PICC line). CVC lines are used to administer medication or fluids that are unable to be taken by mouth or would harm a smaller peripheral vein. They are also used to obtain blood tests and measure central venous pressure.

Present methods of drawing blood from a patient through a CVC include multiple steps where each step introduces a chance of contamination into the patient's blood stream. Additionally the method necessitates the unneeded waste or discarding of blood from the patient. This method uses 3 separate syringes.

During this method, a CVC line is attached to a patient. A first syringe with 10 cc of normal saline is located in the CVC hub and used to flush the CVC line. The term “flush” refers to a solution of saline that is used to clear the CVC line of anything that was being infused in the patient. After flushing, a quantity of fluid is drawn back (i.e. 10 cc of waste) into the syringe. The waste refers to a mixture of patient blood and saline solution involved in the flushing done in the prior step. The first syringe is removed and replaced with a second syringe. The second syringe is located in the CVC hub and draws back between 1 cc and 20 cc of fresh undiluted blood. The second syringe is then removed and replaced with a third syringe containing 10 cc of normal saline. This saline is flushed through the CVC. The third syringe is then removed.

Each time a syringe is removed and replaced, opens up an opportunity for contamination. That is 3 possibilities of introducing a bacteria or virus into the patients' blood stream. Each syringe detaches from and makes contact with the CVC directly. This method also wastes 10 cc of the patients' blood every time blood is drawn, which could be as often as every two hours. So, this method allows two negatives: multiple opportunities for introduction of something undesirable into the patients' bloodstream, and it does not promote blood conservation.

Although strides have been made to provide effective and efficient practices and tools in the medical industry, shortcomings remain. It is desired that an assembly be provided that allows for the selective communication of multiple syringes with a CVC without repeated detachment with the CVC itself. It is desired that the assembly conserve blood of the patient and minimize the chance of infection.

### BRIEF SUMMARY OF THE INVENTION

It is an object of the present application to provide a non rotatable CVC configuration that is configured for use in a non-sterile setting, such as at the bedside of a patient. An object of the CVC line blood drawing assembly of the present application is to be non reusable, or disposable, via a singular removable port. The assembly is meant to be a closed system to eliminate the ability for contamination.

It is an object of the present application to provide a blood drawing assembly for a CVC line. The assembly is configured to utilize a single attachment device for communication with the CVC and selectively alternate communication between a plurality of syringes. This allows a single communication with the CVC hub despite the use of multiple syringes. In fact, multiple syringes are alternated through with a single syringe attachment device in communication with the CVC hub.

It is a further object of the present application that the assembly permit for the return of flushed “waste” back into the patient. Additionally, at least one syringe is removable from the assembly for the purpose of filling vials used to transfer blood to a lab or other facility.

The method of using the assembly involves the single connection of the CVC hub with the attachment device. Operation of the syringes is used in a predefined order wherein the set of syringes are sequentially engaged with the CVC line and brought in selective communication with the attachment device. At least one syringe is fully removable from the base. Ultimately the invention may take many embodiments. This assembly overcomes the disadvantages inherent in the prior art.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a blood drawing assembly according to an embodiment of the present application.

FIG. 2 is a front view of the assembly of FIG. 1.

FIG. 3 is a left side view of the assembly of FIG. 2.

FIG. 4 is a top view of the assembly of FIG. 2.

FIG. 5 is an upper perspective view of a collection hub in the assembly of FIG. 1.

FIG. 6 is a lower perspective view of the collection hub of FIG. 5.

FIG. 7 is a front view of the assembly of FIG. 1 with a collection hub being partially removed to show internals and operation of the assembly.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The assembly and method in accordance with the present application overcomes one or more of the above-discussed problems commonly associated with common CVC line practices discussed previously. In particular, the assembly captures a plurality of syringes in a translating collection hub that is configured to selectively provide for their respective engagement with a syringe attachment device that is in communication with a CVC line and CVC hub. The assembly is able to alternate syringes without detaching the CVC hub from a CVC. The assembly is configured to be a closed system as it is intended for use primarily in non-sterile environments where contamination is an issue. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The assembly and method of the present application is illustrated in the associated drawings. The assembly includes a CVC hub and CVC line extending into a collection hub having a plurality of syringes. The CVC line is coupled to a syringe attachment device within the hub. The device may be translated along a path that allows it to come into selective contact with each of the plurality of syringes coupled to the collection hub. The collection hub includes a plurality of ports for acceptance of the plurality of syringes. The syringes are configured to selectively alternate between a withdrawn position which is below a translating plane of the syringe attachment device and that of a seated position wherein the top of the syringe rises above the translating plane of the syringe attachment device. Selective translation of the device as well as the sequential engagement of the syringes into and out of a seated position allows a user to successfully operate the assembly. Additional features and functions are illustrated and discussed below.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

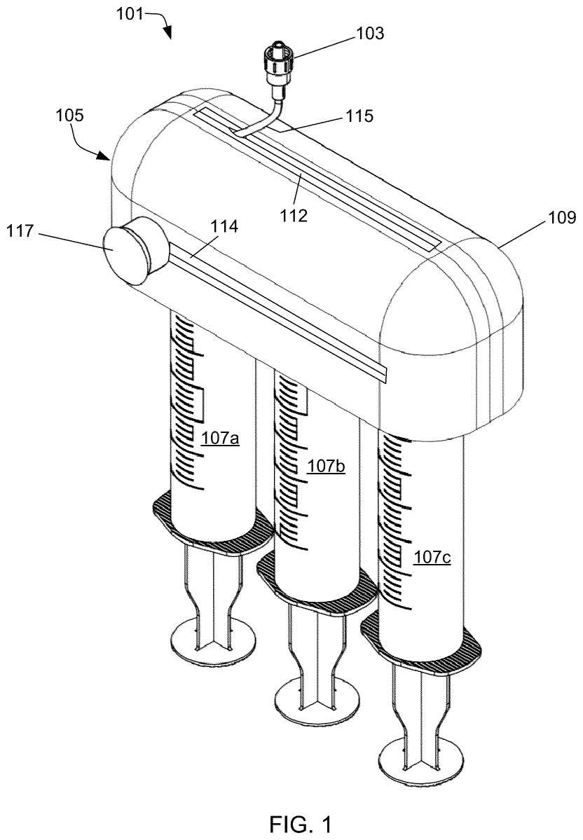

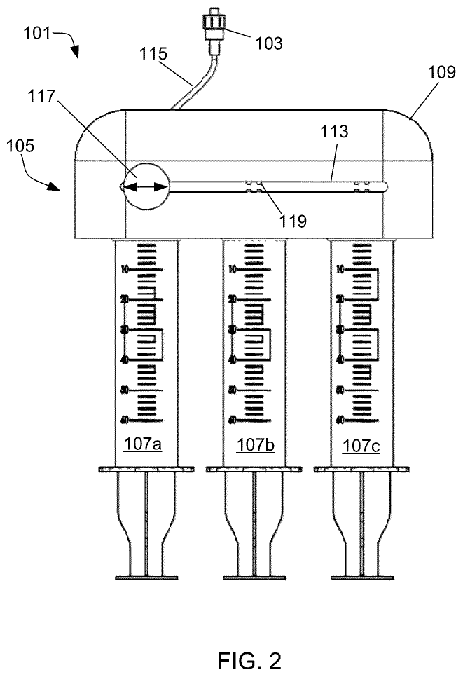

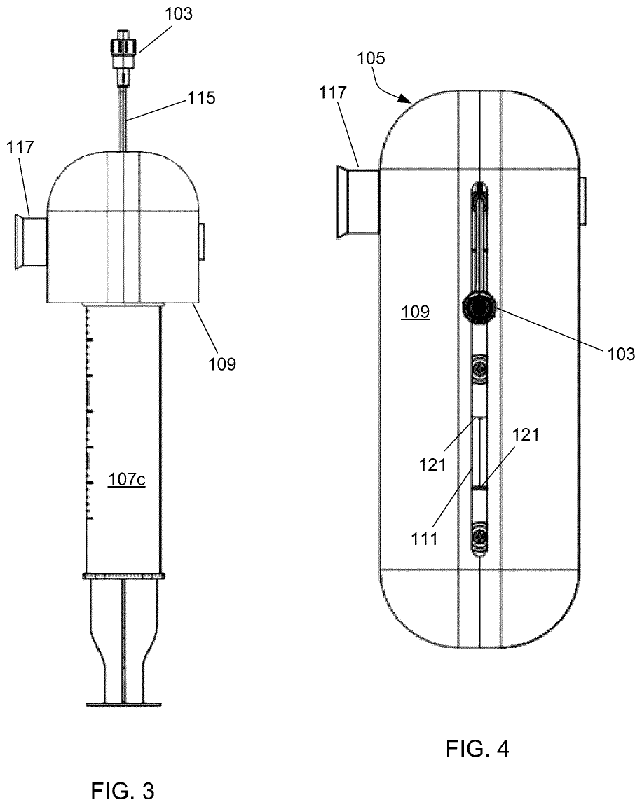

Referring now to FIGS. 1-4 in the drawings, views of a blood drawing assembly 101 is illustrated. Assembly 101 is configured to provide a single and continuous attachment device in contact with a CVC (central venous catheter) to allow for the withdrawal and insertion of fluids into a vein of the human body. The term CVC stands for a central venous catheter that is attached to a patient for the long-term transfer of fluids into or out of the body. Assembly 101 is configured to selectively engage a CVC hub, which connects to the CVC, with a plurality of syringes in a sequential manner, so as to allow fluid communication between a patient's vascular system and at least one of the plurality of syringes at any given time. In other words, blood may pass from the CVC, through the CVC hub, and selectively engage with any number of syringes. This decreases the chance of infection as only a single communication is made with the CVC hub despite the use of multiple syringes.

Assembly 101 includes CVC hub 103, a collection hub 105, and a plurality of syringes 107a/107b/107c. Collection hub 105 includes a body 109 that is hollowed internally. Body 109 includes an upper slot 111 and a lower slot 113 configured to pass through body 109. In FIG. 1, slots 111 and 113 are shown as including membranes 112 and 114. Membranes 112 and 114 are configured to close the slots so as to ensure a sterile barrier. Membranes 112 and 114 are removed from the remaining figures for clarity purposes. It is understood that each membrane may be formed or shaped in any known manner. As seen in FIG. 1, each membrane includes a central slot that is configured to separate with the passage of line 115 or knob 117.

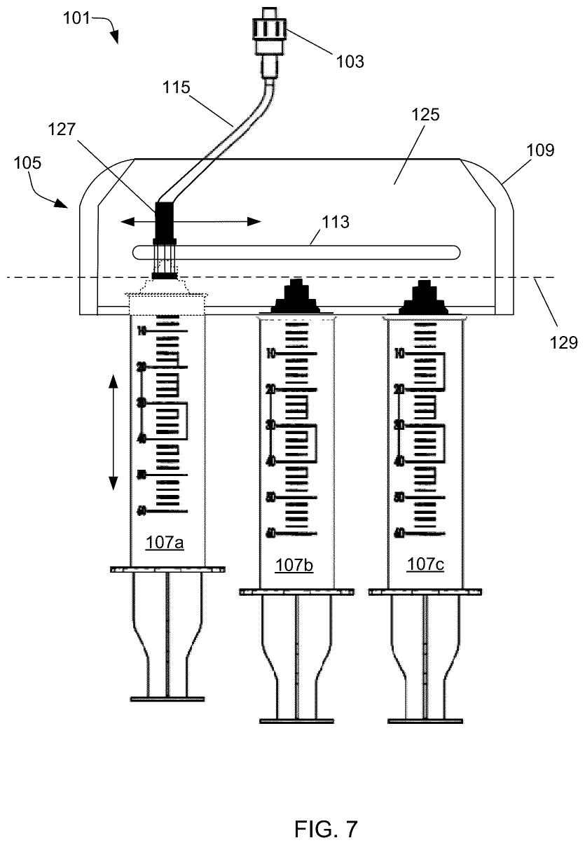

As seen in FIG. 1, the syringes are configured to engage a lower surface of body 109 so as to be partially secured within the internal volume of body 109. Further illustration will be provided in FIG. 7. CVC hub 103 is coupled to CVC line 115 which passes through upper slot 111 and extends into the internal volume 125 of body 109. Fluids may pass through hub 103 and line 115 both into or out of syringes 107a/107b/107c.

It is further understood that collection hub 105 further includes a translating knob 117 which is configured to selectively pass through lower slot 113. Knob 117 is configured to translate along the length of slot 113 so as to engage CVC hub 103 with anyone of the plurality of syringes. Each syringe is seated or located within a particular location of body 109. In order to assist in alignment of CVC hub 103 and CVC line 115 with that of any syringe, slot 113 is configured to include locating tabs 119. Locating tabs 119 are configured to set translating knob 117 in alignment with a syringe, and act to provide temporary securement of the translating knob 117 within slot 113. Multiple locating tabs 119 are used within slot 113. It is understood that locating tabs 113 may be located along a lower surface or upper surface of slot 113.

Of note in particular with FIG. 4 is the ability to see ports 121 formed to accept the syringes. The ports 121 are formed along a lower surface of hub 105. Viewing hub 105 from the top allows one to visualize ports 121 through upper slot 111. As alluded to previously, syringes are configured to engage ports 121 of collection hub 105. Locating tabs 119 are individually aligned with each syringe.

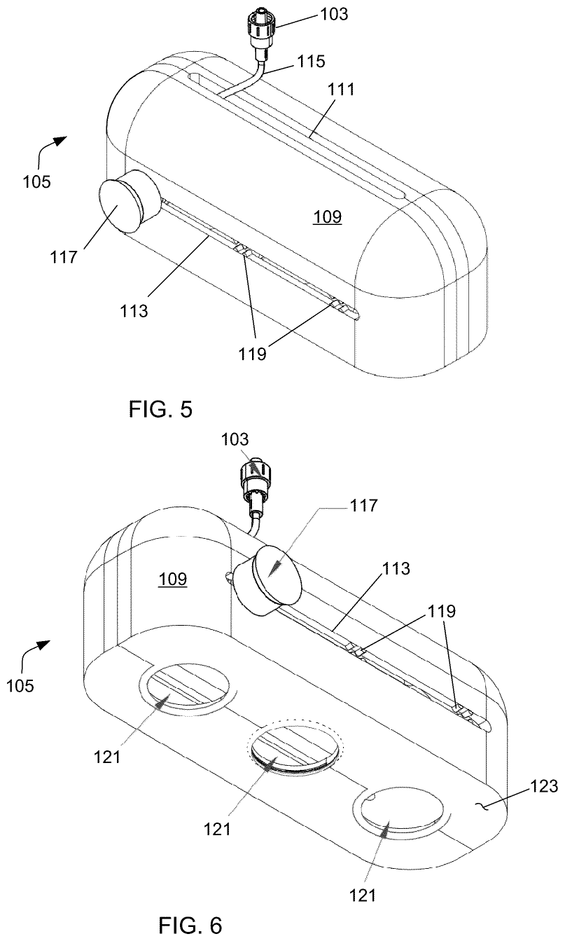

Referring now also to FIGS. 5 and 6 in the drawings, assorted views of collection hub 105 are provided. FIG. 5 illustrates an upper perspective view of collection hub 105 while FIG. 6 illustrates a lower perspective view of collection hub 105. CVC hub 103 is included in the figures to assist with clarity purposes. As seen in particular with FIG. 6, ports 121 are formed along lower surface 123 of hub body 109. Syringes are configured to pass through ports 121 and selectively engage with hub body 109.

It should be reiterated that the syringes are ideally affixed to collection hub 105 so as to prevent their removal. A singular syringe may be detachable from hub 105 to facilitate blood drawing within multiple vials. By coupling syringes (i.e. syringe 107a and 107b) to collection hub 105 in a non-removable manner, the ability for contamination from the nonsterile environment is drastically decreased. As stated previously, assembly 101 is a non reusable assembly. The syringes along with collection hub 105 will be discarded after each use. Although other devices are capable of collecting blood from a CVC, such are typically only useful in sterile environments such as an operating room but are not sufficient for use in non-sterile environments such as at the bedside of a patient in their home.

It is also worth noting that location tabs 119 are also illustrated along an upper surface of slot 113. It is understood that hub 105 may include any one of a number of attachment methods to secure syringes to body 109. These may include interference fit, clasps, threaded engagement, or even a webbing of material across the opening of ports 121. Hub 105 is configured to selectively secure and hold a plurality of syringes within ports 121 pending selective engagement with CVC hub 103.

Referring now also to FIG. 7 in the drawings, a front view of assembly 101 is illustrated with body 109 partially removed to show an interior volume therein. FIG. 7 is partially useful to provide an illustrative look at the operation of assembly 101. As noted above, body 109 is partially withdrawn so as to illustrate an internal volume 125. Lower slot 113 remains in FIG. 7 to provide elevation and context for the location of slot 113 relative to each syringe in operation. Locating tabs 119 have been removed.

Collection hub 105 further includes a syringe attachment device 127 that is coupled at a first end to CVC line 115. CVC hub 103 is coupled to CVC line 115 at a second end. Fluids are configured to pass through hub 103 and line 115 into device 127. It is understood through the Figure that each syringe is configured to translate relative to collection hub 105. This translation allows each syringe to selectively engage with or disengage with device 127. As shown in FIG. 7, syringe 107a is located in a seated position wherein syringe 107a is elevated or raised into internal volume 125 so as to engage device 127. At the same time syringes 107b and 107c are located within ports 121 but remain in a withdrawn position. A translating plane 129 illustrates a distinction between the withdrawn position and the seated position. When each syringe is in a withdrawn position, the syringes are below translating plane 129. In this position, device 127 is enabled to slide or translate horizontally between each of the syringes. Locating tabs 119 are provided at each syringe location to help orient device 127 in proper alignment. Translating knob 117 is coupled to device 127 and provides a user an interface for locating device 127 within internal volume 125.

In operation of assembly 101, it is desired that one or more of syringes 107a/107b/17c are brought into selective contact or communication with device 127 at a time. CVC line 115 is configured to move in slot 111 with device 125 as dictated by the movement of knob 117. A number of syringes may be prefilled with a saline solution to assist in flushing the line and CVC for the collection of a blood sample. Once engaged with device 127, a syringe's plunger may be operated to introduce saline into CVC hub 103. Likewise, retraction of the plunger may be used to withdraw blood or saline and blood solution. When one syringe is left free for removal from hub 105, that syringe may be operated to remove blood from hub 103. Other syringes may be attached in place of that syringe as needed. Saline is useful for flushing the line from whatever has been provided to the patient so as to not contaminate any blood sample needed at that time.

A sample operation may look as follows. A medical professional scrubs the CVC hub and opens a sterile packaging with assembly 101 therein. CVC hub 103 is coupled to the patient and syringe 107a is engaged with the syringe attachment device. The medical professional operates the plunger of syringe 107a to collect waste fluid within the CVC. The medical professional disengages syringe 107a and operates translating knob 117 to align with syringe 107b. Syringe 107b is engaged with the syringe attachment device. The plunger of that syringe is operated to collect a blood sample from the patient. Syringe 107b is disengaged from the syringe attachment device. The medical professional switches the syringe attachment device back to syringe 107a and engages it. The medical professional depresses the plunger of syringe 107a that still contains the waste fluid, giving that fluid back to the patient thereby promoting blood conservation. Syringe 107a is disengaged from the syringe attachment device and the medical professionals translates knob 117 into alignment with syringe 107c. Syringe 107c is engaged with the syringe attachment device and the plunger is depressed dispensing 20 cc's of normal saline solution through CVC line 115 and CVC hub 103. The saline solution is pre-instilled within syringe 107a. The medical professional removes assembly 101 from the CVC. The medical professional may remove syringe 107b to collect that blood sample. Syringe 107b may be used to fill one or more vials of blood for testing.

An important feature of the present assembly is the ability to be one-time use, or discardable. A plurality of syringes are affixed or non removable from hub 105. This is done to eliminate potential contamination from the non-sterile environment. A number of procedures may necessitate blood samples from a patient but not all patients are located in a sterile environment like an operating room. The assembly is configured to provide a clean manner of sampling a patient while being in a non-sterile environment.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A CVC blood drawing assembly, comprising:
a CVC hub and a CVC line, the CVC hub coupled to a second end of a CVC line;
a collection hub having a body and an internal volume, the first end of the CVC line routed into the internal volume through an upper slot in the body;
a syringe attachment device located within the internal volume and coupled to the first end of the CVC line; and
a plurality of syringes located within a plurality of ports along a lower surface of the collection hub, at least one of the plurality of syringes is affixed to the collection hub so as to be non-removable;
wherein the plurality of syringes are selectively brought into engagement with syringe attachment device while the CVC hub remains coupled to the CVC.

2. The assembly of claim 1, wherein the body is configured to secure against at least one of the plurality of syringes to prevent removal.

3. The assembly of claim 1, wherein the plurality of syringes operate between a withdrawn position and a seated position.

4. The assembly of claim 3, wherein the plurality of syringes are configured to selectively translate back and forth within the hub body when transitioning between the withdrawn position and the seated position.

5. The assembly of claim 1, further comprising:
a lower slot in the body; and
a translating knob in communication with the syringe attachment device in the internal volume, the translating knob configured to pass through the lower slot and locate the syringe attachment device in line with any of the plurality of syringes.

6. The assembly of claim 5, wherein the lower slot includes a membrane to form a sterile barrier for the internal volume.

7. The assembly of claim 5, wherein the upper slot includes a membrane to form a sterile barrier for the internal volume.

8. The assembly of claim 5, wherein the lower slot includes a locating tab configured to align the syringe attachment device with the plurality of syringes.

9. The assembly of claim 1, wherein only one of the plurality of syringes is removable from the base member.

10. A method of exchanging fluids through a CVC, comprising:
coupling a CVC hub to the CVC;
routing a CVC line from the CVC hub to a collection hub, the collection hub including a syringe attachment device and a plurality of syringes coupled thereto;
aligning the syringe attachment device with a first syringe in the plurality of syringes;
engaging the syringe attachment device with the first syringe by moving the first syringe into the collection hub; and
selectively engaging the plurality of syringes with the syringe attachment device for the exchange of fluids;
wherein the CVC hub remains in contact with the CVC as the plurality of syringes alternate selective engagement with the syringe attachment device.

11. The method of claim 10, wherein at least one of the plurality of syringes is removable from the collection hub.

12. The method of claim 10, wherein at least one of the plurality of syringes is non removable from the collection hub.

13. The method of claim 10, further comprising:
reinjecting waste fluid back through the attachment device from at least one of the plurality of syringes.
